(19) United States
(12) Patent Application Publication
(10) Pub. No.: US 2024/0038377 A1
(43) Pub. Date: Feb. 1, 2024

(54) DENTAL EQUIPMENT SERVICE APPLICATION

(71) Applicant: A-DEC, INC., Newberg, OR (US)

(72) Inventors: Robert Michael AYERS, Newberg, OR (US); Ryan Michael WILLIAMS, Newberg, OR (US); Lee AN, Newberg, OR (US)

(73) Assignee: A-DEC, INC., Newberg, OR (US)

(21) Appl. No.: 17/875,924

(22) Filed: Jul. 28, 2022

(51) Int. Cl. G16H 40/40; G06F 16/53

(52) U.S. Cl. CPC G16H 40/40; G06F 16/53

(57) ABSTRACT

A dental equipment service application that allows for capture of an image of a component of a dental equipment, and using the image to search for a replacement, is described. In embodiments, the image is used to search a database of replacement parts, such as with a machine learning algorithm. Possible replacement parts may be provided via an API to facilitate purchase. The application may further allow access to diagnostic information on the equipment and documentation relevant to the replacement parts. Other embodiments may be described and/or claimed.

### TECHNICAL FIELD

Disclosed embodiments are directed to an application for providing service to dental equipment, and specifically to an application that allows for integrated and holistic equipment servicing that includes parts searching, ordering, diagnostics, and documentation.

### BACKGROUND

Dental offices use a variety of specialized equipment, necessary to the dentistry profession. Equipment may include adjustable chairs, dental lights, sterilizers, a variety of dental implements such as rotary handpieces, ultrasonic scalers, lasers, intraoral cameras, digital scanners, suction instruments, and cuspidors, and supporting mechanical equipment such as dental units, vacuum pumps, compressors and air purifiers. These various pieces of equipment may include both mechanical components and electronic components, which may be used to control and coordinate function of the various mechanical components. As is generally the case with machines, various components may be subject to wear, degradation, and/or failure, even with routine maintenance.

When equipment fails, typically someone on staff at the dental office must get in touch with a service technician, either from the equipment manufacturer of with a qualified service provider. The service technician will typically visit the dental office to assess the equipment and effect any necessary repairs, including obtaining any needed replacement parts. As a dentist typically requires the equipment to be in working order to care for patients, it is critical that equipment be repaired and brought back into service in as expedient a fashion as possible.

The background description provided herein is for the purpose of generally presenting the context of the disclosure. Unless otherwise indicated herein, the materials described in this section are not prior art to the claims in this application and are not admitted to be prior art by inclusion in this section.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIG. 1 illustrates an example system within which a dental equipment service application may be utilized, according to various embodiments.

FIG. 2 is a flowchart of the various operations of a method that a dental equipment service application may execute to facilitate service of dental equipment, according to various embodiments.

FIG. 3 is a block diagram illustrating various functional aspects of an example dental equipment service application, according to various embodiments.

FIG. 4 is a block diagram of an example computer that can be used to implement some or all of the components of the disclosed systems and methods, according to various embodiments.

FIG. 5 is a block diagram of a computer-readable storage medium that can be used to implement some of the components of the system or methods disclosed herein, according to various embodiments.

### DETAILED DESCRIPTION

In the following detailed description, reference is made to the accompanying drawings which form a part hereof wherein like numerals designate like parts throughout, and in which is shown by way of illustration embodiments that may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the accompanying description. Alternate embodiments of the present disclosure and their equivalents may be devised without parting from the spirit or scope of the present disclosure. It should be noted that like elements disclosed below are indicated by like reference numbers in the drawings.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

In some figures, multiples of a same or similar type of element may be designated with a, b, c, etc., such as 100a, 100b, 100c. Such elements may be collectively or generically referred to by just the call-out number, e.g. 100, with a letter added when a specific one of the element is intended.

As used herein, the term “circuitry” may refer to, be part of, or include an Application Specific Integrated Circuit (ASIC), an electronic circuit, a processor (shared, dedicated, or group) and/or memory (shared, dedicated, or group) that execute one or more software or firmware programs, a combinational logic circuit, and/or other suitable components that provide the described functionality.

Service technicians called upon to repair dental equipment will typically need to assess a failed or malfunctioning piece of equipment to ascertain which component or components have failed. Such an assessment usually requires the technician to visit the facility where the equipment is installed and perform various diagnostic steps to locate any malfunctioning components. In some cases, multiple pieces of equipment may need to be evaluated. For example, if an air-driven implement such as a rotary handpiece has failed, the reason for failure could be located in the handpiece, its electronic controller, the dental unit conveying compressed air to the handpiece, and/or in the compressor itself. Each piece of machinery may be relatively complex with multiple components, and accordingly multiple possible points of failure.

Given the complexity of many modern pieces of dental equipment, it is usually not feasible for a service technician to stock replacements for all possible components that could fail for even a given make and model of equipment, let alone all makes and models of equipment that a given technician may be called upon to service. If a failed component is not one that is commonly stocked by a service technician, such a component may need to be ordered from a parts supplier. Ordering the correct part itself may pose a challenge when different variations of a component are available that are specific to a given make and model of dental equipment. Furthermore, different pieces of dental equipment may have different procedures for replacement and installation of failed components. Commensurate with a wide number of makes and models of equipment, it may be infeasible for a service technician to be readily familiar with the repair and/or replacement steps for every possible component the technician may be called upon the service.

For a given piece of equipment, a service technician typically has one or more specific components physically in front of them that they wish to understand, to determine subsequent action. Often, they wish to identify the part number of the component, such that they can order a replacement during repair and/or find other useful reference information for troubleshooting or estimating costs. Currently, this process can be a challenge, consisting of Internet searches and/or attempting to locate a service parts catalog, service guide, or other printed resource. Even if a relevant document is located, the information may be outdated, and they may need to spend further time attempting to find the part number or part numbers corresponding to the component being serviced or replaced.

Additionally or as an alternative, a service technician may reach out to customer service of the equipment manufacturer for help. This communication, however, may consume additional valuable time. Because the customer service center typically has no knowledge of the service technician's efforts, most calls begin with the technician explaining who they are, the basics of the issue, and the details of the problem or equipment failure. This information typically cannot be shared with the customer service representative in an automated fashion. Consequently, existing service workflows rely upon manual methods to understand issues and subsequently process necessary outcomes, due at least in part to the existence of various different siloed systems.

Accordingly, there is tremendous value in having information about a particular service call be available from one, user-friendly single source of truth. Such a central source of information could enable a service technician to access whatever information they need in a timely fashion, preferably during or before, such as via remote monitoring, the initial service call to the dentist office.

In disclosed embodiments, data can be captured and entered by an application used by a service technician, such as an application running on a mobile device or accessible via a web browser. Such data may include an image of a component in need of service, and/or diagnostic information that may be accessed and retrieved from the equipment being serviced as well as any supporting equipment. This information could then automatically be integrated into other systems to enable faster and more tailored support outcomes from both manufacturer customer support and/or other support partners, e.g. dealers, parts providers, etc. For example, identified parts needing replacement could more seamlessly be integrated into dealer ordering systems via API connections. Furthermore, in embodiments an identified part could launch specific technical reference information relative to that component or submit a field input report for a complaint, provide equipment information and/or automatically take any other appropriate support actions.

As suggested above, many pieces of dental equipment may have on-board diagnostic capabilities, such as providing the status of various machine parameters, fault detections, etc. In some cases, this diagnostic data may suggest to a service technician components that are in need of service or replacement. In the case of most equipment, such diagnostic information is only accessible locally, such as on the actual piece of equipment; there is no method to examine machine status and diagnostic data when a service technician is located at a remote location away from the equipment. Thus, a technician would need to spend valuable time traveling to a physical product to troubleshoot the equipment on site, which involves interrupting the workflow of the dentist. As mentioned above, the service technician may then need to order any replacement parts, and await their arrival before returning to the dental office to complete repairs. This results in a higher number of ‘truck rolls’ or visits by the technician, and may decrease the number of offices that a given technician can service within a given timeframe.

Conversely, being able to easily remotely access the diagnostic information of a given piece of equipment may allow the service technician to come better prepared with likely repair parts during their initial visit. Furthermore, if the service technician needs to engage with the manufacturer's customer service, the process to describe the problem and subsequent technical details may be at least partially automated, further saving time. Customer service personnel may be able to remotely access the equipment diagnostic information when a support call is initiated, to recommend appropriate next steps more accurately and efficiently. Disclosed embodiments may further allow diagnostic information to be remotely accessed.

Other possible variations may include predictive analytics, which may be based upon such remotely accessible diagnostic information. Captured data from sensors and electronics within equipment could be streamed to the cloud or other remote service facilities, where data analysis such as trending and/or more sophisticated data analysis techniques could be performed. Artificial intelligence and other more complicated algorithms could be applied with the aim to apply more advanced predictive analytics.

Disclosed embodiments provide a holistic service application. Other aspects of this holistic service application, in various embodiments, may include the potential for seamless integration into customer relationship management tools to enable more efficient technical support, where inputs from the application are already visible to the service agent upon answering a call. Other embodiments may include the capability to perform remote guided troubleshooting conducted in a live fashion over a video call by remote experts.

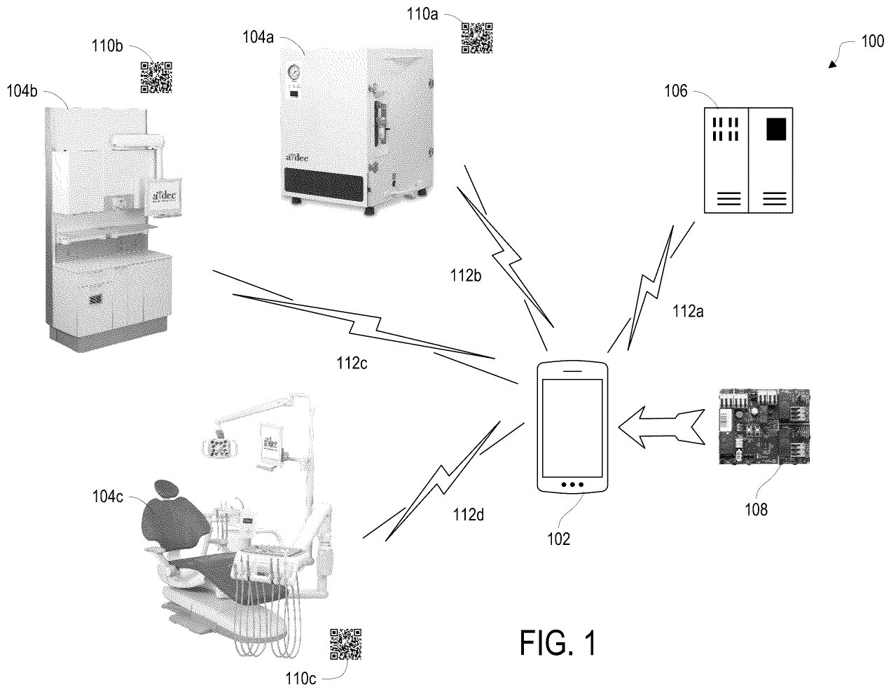

FIG. 1 illustrates an example system 100 with which a dental equipment service application, according to various embodiments, may be employed. System 100 includes a device 102, such as a mobile device, which may be in data communication with one or more pieces of dental equipment 104, such as compressor 104a, dental furniture 104b, and dental chair 104c, for a few examples. Device 102 may further be in data communication with a remote system 106.

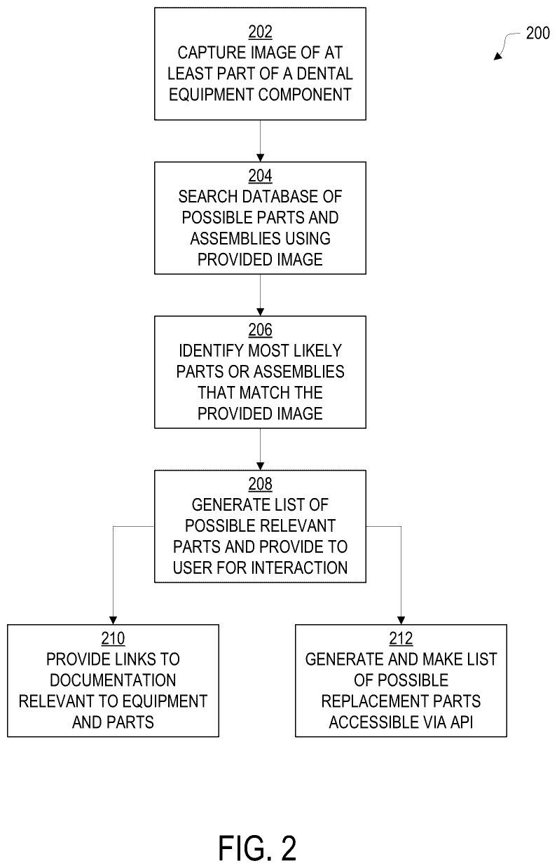

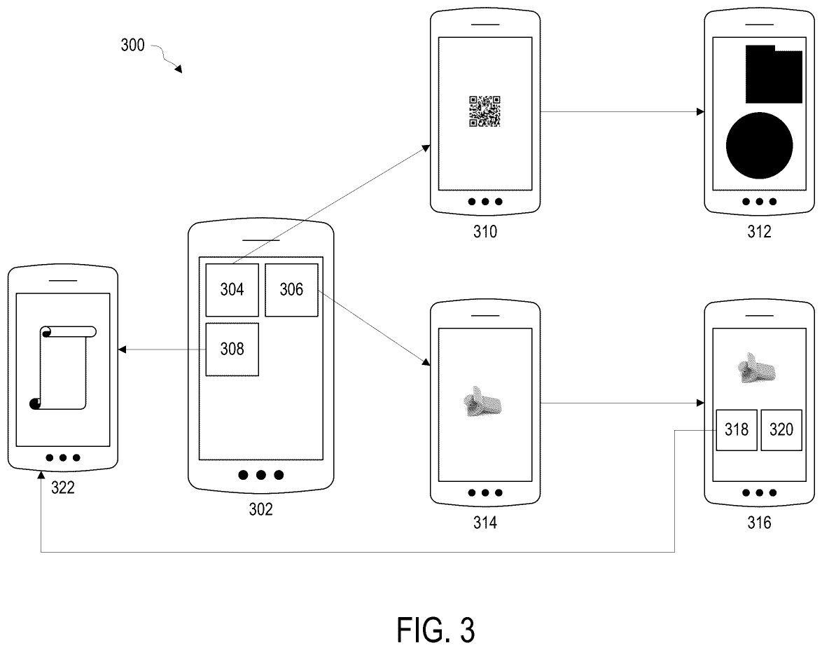

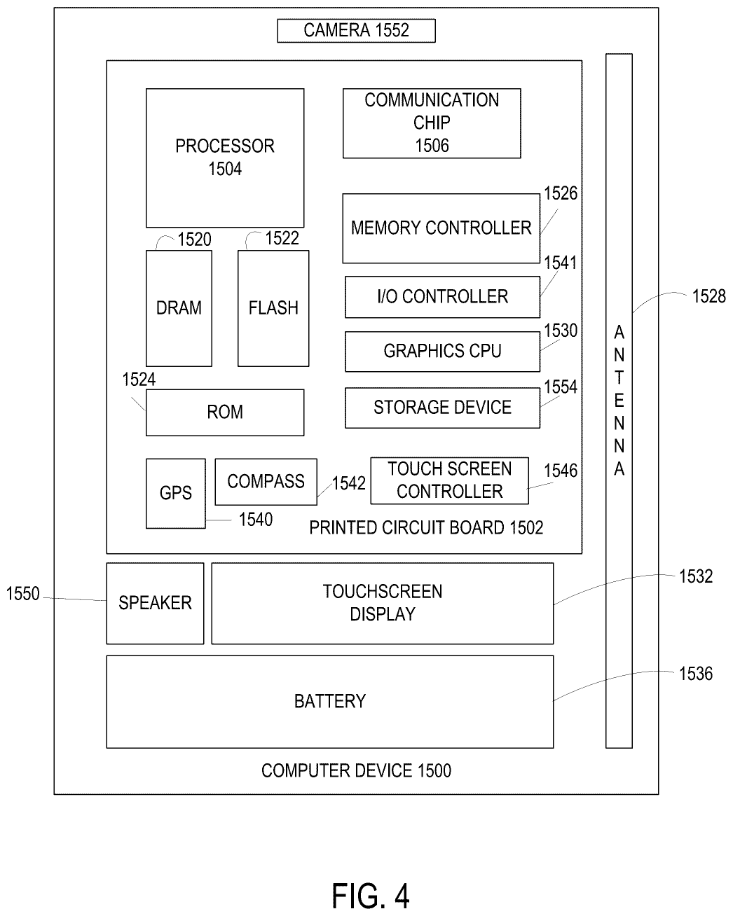

Device 102 may be a mobile device, such as a smartphone, personal digital assistant, or tablet, or may be a more fully-functioned device such as a laptop, desktop, or other suitable computer system. Device 102 may be implemented as a computer device 1500, described below with respect to FIG. 4. Device 102 may include or be connected to a camera or other image capture device, so that images can be captured and stored into device 102. Device 102 may further be configured to communicate with dental equipment 104 and/or remote system 106 via a wired or wireless data communications link, as is known. Device 102 further is configured to run a dental equipment service application, such as will be described below with respect to FIGS. 2 and 3, which may be provided on a non-transitory computer readable medium, such as storage medium 1602 with instructions 1604.

Remote system 106 may be implemented as a remotely located server or cluster of servers, or may be implemented as part or all of a data center or cloud service. As will be discussed herein, in embodiments remote system 106 may implement at least part of the functionality offered by or otherwise accessible through the dental equipment service application that runs on device 102. In embodiments, remote system 106 may be owned and/or operated by the manufacturer of dental equipment 104. It further should be understood that although system 100 is illustrated as only having a single remote system 106, device 102 may be in contact with multiple different remote systems 106, such as where various pieces of dental equipment 104 are provided by different manufacturers. Alternatively, each manufacturer of a dental equipment 104 can be thought of as having a separate implementation of system 100, with a single device 102 interfacing across multiple implementations of system 100.

In operation within system 100, in embodiments, a dental equipment service application running on device 102 may cause device 102 to capture an image, such as an image of a component 108. Component 108 may be a component of any one of dental equipment 104. The component may be installed in the dental equipment or uninstalled at the time the image is captured. The component may be a single part or an assembly of multiple parts up to and including an entire dental equipment product, or set or system of products. In some embodiments, a component may itself include multiple components, e.g. subassemblies or sub-components. Thus, a component may be any single part or assemblage of parts and/or components. The image 108 may then be supplied to remote system 106 for further analysis, in some embodiments.

In embodiments, the dental equipment service application may also cause device 102 to capture an image of an optical marker, such as one of optical marker 110a, 110b, or 110c. As depicted, each optical marker 110 is associated with a corresponding dental equipment 104, viz. optical marker 110a is associated with dental equipment 104a, optical marker 110b is associated with dental equipment 104b, and optical marker 110c is associated with dental equipment 104c. Each optical marker 110 may encode information about registering and communication with its associated dental equipment 104 that is readable by device 102, to allow device 102 to connect to the associated dental equipment 104. In some embodiments, the encoded information may include a unique ID code, such as the equipment serial number, or another suitable code that can uniquely identify the dental equipment 104 for registration and communication.

The optical marker 110 is depicted as a QR code in FIG. 1, but it should be understood that any optical marker that can encode the appropriate information about its associated dental equipment 104 may be employed, such as a bar code or similar optical encoding of the serial number of the equipment 104, which may be affixed to the equipment 104. Furthermore, in other embodiments, a radio beacon such as an NFC tag, RFID tag, or Bluetooth® beacon may be employed to provide the encoded information and/or to facilitate registration and a data connection between device 102 and a dental equipment 104. In still other embodiments, any other suitable method of providing device 102 with necessary connection information may be employed, which may include manual entry of the encoded information or unique ID by a user. Each optical marker 110 may be physically attached to its corresponding dental equipment 104 at an appropriate location. In other embodiments, optical marker 110 may be provided by the equipment manufacturer, such as via a website or application, where it may be looked up by reference information such as equipment model and/or serial number, possibly in conjunction with information about the equipment 104's registered owner.

The optical marker 110 (or radio beacon or other implement) may encode information allowing device 102 to communicate by a data link 112. As seen in FIG. 1, a data link 112 may be established between each dental equipment 104 as well as remote server 106. Depending upon the embodiment, the data link 112 may allow unidirectional (one-way) or bi-directional (two-way) communication with device 102. As will be discussed herein, each respective data link 112 may be used to exchange diagnostic information from its associated dental equipment 104. With respect to remote server 106, data link 112a may be used to provide remote server 106 with diagnostic information obtained from one or more of the dental equipment 104 and/or an image 108 of a component from one of the dental equipment 104. Furthermore, data link 112a may be used by device 102 to receive information from remote server 106, such as parts lists and/or documentation, as will be described below with respect to FIGS. 2 and 3. It should be understood that, in embodiments, each data link 112 may be transitory, being established and used only when necessary, and being disconnected when dormant; in other embodiments, one or more data links 112 may be continually established.

Although not illustrated, in other embodiments, one or more of the dental equipment 104 may be in direct communication with remote server 106, and may not need to use device 102 as a relay for diagnostic information. For example, remote server 106 may be accessible by a customer service agent or center operated by the manufacturer of a given equipment 104, which may allow the customer service agent or center to directly access diagnostic information provided by equipment 104 even if a user of device 102 has not yet established a data link 112 with the equipment 104. In other embodiments, diagnostic information may be obtained from one or more sensors equipped to equipment 104, such as temperature sensors, vibration sensors, current sensors, voltage sensors, pressure sensors, vacuum sensors, humidity sensors, and/or any other sensors appropriate for measuring the health of a given equipment 104. Information from these sensors may be analyzed, such as using artificial intelligence and/or another suitable analytical method, to provide predictive analytics and trends. For example, such analytics may be able to provide a preliminary diagnosis of equipment condition and possible ongoing or future failures. Such preliminary diagnosis may further be used to automatically generate a preliminary list of possible replacement components and/or to generate or prompt for creating a service call appointment.

In FIG. 2, the operations of an example method 200 that may be carried out by a dental equipment service application are depicted, according to various embodiments. In the illustrated embodiment, the operations of method 200 allow the dental equipment service application to visually determine any parts or assemblies, collectively referred to as components, of a piece of dental equipment that is being serviced. Method 200 may be implemented on a system such as system 100, and may be carried variously by a device such as device 102 and/or remote server such as remote server 106. The various operations of method 200 may be carried out in whole or in part, and may be carried out in a different order than as depicted, depending upon the needs of a given implementation.

In operation 202, an image of a component from a dental equipment, such as image 108, is captured. The image may be captured directly, such as where the capturing device is a mobile device such as smartphone, or otherwise has a camera directly attached. In some embodiments, the image may be of only a portion of component, and/or the image may depict a component that is damaged, dirty, discolored, or otherwise somewhat different from a new component.

In operation 204, the image is used to search into a database of possible parts and assemblies of various dental equipment. In some embodiments, operation 204 may be carried out at least partially by a remote server, such as remote server 106, by the capturing device uploading the image to the remote server. The remote server may then implement a suitable algorithm for conducting a search of images using the image as a search key. In other embodiments, the search may be performed at least in part, or a search formulated by, the capturing device.

The remote server may implement the search algorithm using a machine learning technique. For example, a suitable neural network, such as a deep neural net, convolutional learning net, or another type of neural net may be trained using a collection of images of all stored replacement parts in various perspectives. Depending on the types of training sets employed with the neural net, the facility of the search algorithm to accurately match a replacement part or parts from an incomplete or somewhat obscured image of the dental equipment component may be improved. Multiple images for a given part can improve matching regardless of the perspective of the component presented in the image used for the search, and may allow a match even if the component is damaged, dirty, or otherwise obscured.

In operation 206, in embodiments, one or more parts or assemblies are identified as the result of the image-based search performed in operation 204. The supplied image may be of a single dental equipment component, which itself may consist of multiple parts. Hence, the search may return multiple possible parts that comprise the component. While various embodiments contemplate the imaged part as being used, defective, or otherwise in need of service, it should be understood that method 200 could equally be applied to new components, such as to facilitate access to documentation relevant to the part, rather than or in addition to obtaining a replacement or spare. In other embodiments, a specific part may be identified from the component as needing replacement, e.g. if a particular part is recognized as broken, burned, or otherwise damaged by differing in appearance from a new part. In still other embodiments, a particular part may differ in appearance from the image captured of the dental equipment component due to redesign or differing source. In such implementations, the neural net or other search algorithm may be trained to match on multiple different versions of the component, and return the most current and compatible part.

In operation 208, a list of suggested part candidates which best match the submitted image is generated from the identified parts from operation 206, in embodiments. The list may be generated by the remote server, or the search results may be provided to the capturing device, which may then generate the list. In some embodiments, a user of the capturing device may be provided a list of possible and/or recommended part candidates, and allowed to either select the parts of specific interest to generate a list, or edit a suggested list. The list of parts may include various relevant information, such as an image of the part, a part number, any associated components and/or subassemblies, part name, compatible equipment, part revision number, other related parts, and/or any other relevant information. In some embodiments, the search results from operations 204 and 206 may only result in a single part, e.g. if the searched part is a single part with no sub-components or related assemblies, in which case operation 208 may be unnecessary.

Following operation 208, links and/or downloads of documentation relevant to the selected and/or identified parts, and/or associated dental equipment, may be provided in operation 210. For example, documentation describing how to locate, remove, install, service, and/or repair a given component with a given part may be made available on the basis of an identified replacement part. Further, information about maintenance and operation of the associated dental equipment may be provided. Other information may include part or equipment specifications. Diagnostic information may be provided to allow a service technician to verify that a given replacement part will address a particular problem, and if not, possible links to other possible candidate replacement parts may be supplied.

In some embodiments, documentation may be accessible to a dental services equipment application without need to search for a replacement part. For example, establishing a connection with a piece of dental equipment, such as described above with respect to system 100 of FIG. 1, may allow information generally relevant to the connected piece of dental equipment to be accessed. Such information may include anything relevant to the operation and/or maintenance of the equipment, e.g. service manuals, repair manuals, maintenance manuals, installation manuals, operation manuals, etc.

In operation 212, the list of possible replacement parts, which may be limited to those parts a service technician has determined are needed, may be added to a “request list”. This request list, in turn may be used or communicated in a number of different ways. For example, the request list may be published to an application programming interface (API), in embodiments, or another suitable communication channel, such as e-mail or another messaging system. The API or communication channel may be publicly accessible, such as via communication with the remote server, so that parts manufacturers and/or suppliers can be notified of the needed part, and ordering of the part can be facilitated. For example, in some embodiments a service technician may be able to use the dental equipment service application to specify and order a given identified replacement part. This order may then be dispatched to an appropriate parts supplier for shipment to the service technician. In some embodiments, the parts supplier may be a supplier with which the service technician (or their hiring company) has an established relationship, e.g. a preferred vendor. In other embodiments, the parts supplier may be an authorized supplier or original equipment manufacturer (OEM) designated by the manufacturer of the dental equipment.

In still other embodiments, the parts supplier may be the dental equipment manufacturer. In some embodiments, the API may be hosted by the remote server, which may be owned and/or operated by the dental equipment manufacturer. In some such embodiments, the API may be accessible by systems hosted by the equipment manufacturer, such as a customer relationship management (CRM) system, service system and/or sales system accessible by the manufacturer's technical support, service or sales department. Alternatively or in addition, the API may be accessible by systems hosted by a third party that sells the dental equipment and/or provides technical service and support concerning the dental equipment, such as a dental sales and service dealer or a parts provider. The CRM, service or sales system may be able to access the API to retrieve or otherwise receive the request list. In other embodiments, the CRM, service or sales system may receive the request list directly, such as via an e-mail inbox. Any suitable method of providing the request list to the CRM, service or sales system may be employed, depending on the specific requirements of a given implementation.

FIG. 3 is a block diagram of various functions of an example dental equipment service application 300, according to embodiments. Starting with an initial screen 302, various controls to access application functionality are provided. Controls may include a button 304 for registering and connecting with a piece of dental equipment to obtain diagnostic information, a button 306 for capturing an image of a component and searching for replacement parts using the image, and a button 308 for accessing relevant documentation. While the depicted interface uses a plurality of buttons, it should be understood that any suitable interface or control that allows access to the associated functionality may be employed, depending upon the needs of a given implementation.

A user of the example application 300 depressing or otherwise activating button 304 may be taken to a screen 310 where an optical marker, such as optical marker 110 depicted in FIG. 1, is captured. The application 300 may then decode the marker to obtain registration and/or connection information with the dental equipment associated with the optical marker, such as equipment 104 depicted in FIG. 1, and establish a connection with the equipment. Following connection, in screen 312 various diagnostic and machine status information may be presented via application 300. This information may be used by a user of application 300 to aid in diagnosing the connected equipment. Further, as described above, this information may be transmitted to a customer service center, in some embodiments. As also described above, in other embodiments, a radio beacon may be used rather than an optical marker to obtain the necessary registration and/or connection information. For example, Bluetooth or NFC may be used to obtain the registration and/or connection information, which may be useful if the optical marker is not available for some reason, e.g. a screen that would produce the marker is broken, non-operable, or otherwise unavailable.

Once connected, button 304 may also allow for access and drilling into diagnostic information of a registered/connected piece of equipment. In some embodiments, analytics such as trends and predictive analytics may be made available. For example, application 300 may either obtain raw diagnostic data and be programmed to provide analysis, and/or may obtain analytics from a remote server, for a given piece of equipment. These analytics may further be used to assist in creation of replacement parts lists and/or in providing access to relevant documentation, such as diagnostic and service manuals.

A user depressing or otherwise activating button 306 may be taken to a screen 314, where an image of a component or part, such as image 108 depicted in FIG. 1, is captured. This image may then be used as a search key into a database, as described above with respect to method 200 of FIG. 2. Following search, a screen 316 with a potential replacement part is depicted. Where multiple possible replacement parts are identified, a user may be able to interact with application 300 to access multiple screens 316 for each identified part, in some embodiments, or by scrolling to view all identified replacement parts, in other embodiments.

In the example screen 316, the identified most likely part(s) or assembly(ies) may be depicted, along with any other relevant information such as a part number, part number description, any needed quantities, any other associated parts that may also be needed to complete a replacement, whether the part is directly orderable, whether an additional or higher-level service kit would be required to purchase, and/or any other relevant information, as described above in method 200. The depiction of the replacement part may be interactive, such as a 3D model that can be rotated, so that a user of application 300 can verify the part is correct, and verify its orientation. Example screen 316 may further include two or more controls, such as buttons 318 and 320. Button 318 may link to relevant documentation, described below with respect to screen 322. Button 320 may add the part to a list of replacement parts to be ordered. Actuating button 320 may further cause the part to be submitted to a public API, such as the API discussed above with respect to method 200, that will allow it to be automatically ordered from a parts supplier.

In still other embodiments, the identified part may be used to provide a link to relevant diagnostic information from the associated dental equipment and/or diagnostic information from the part itself. For example, if a particular board for a dental equipment is identified, the application 300 may provide a link to view or drill into diagnostic information collected from the board. A user of application 300 may thus be enabled to better diagnose any failure or malfunction of the board, which may be correctable or may allow the user to determine if the board needs replacement. Alternatively or additionally, diagnostic information may be obtained from the dental equipment, which may indirectly provide information from and/or about the board.

Actuating either button 308 or button 318 may take a user of application 300 to screen 322, where links to relevant documentation may be provided. As discussed above with respect to method 200, the documentation may be provided via link and/or may be available for download, depending upon the specifics of a given embodiment. In some embodiments, the documentation may be viewable within the application. Still further, depending on whether button 308 or button 318 is actuated, either information general to the dental equipment, for button 308, or specific to the identified part, for button 318 may be supplied. Optionally, information general to the dental equipment may also or alternatively be supplied if button 318 is actuated.

While not depicted, application 300 may offer further functionality beyond that described with respect to FIG. 3. For example, in some embodiments application 300 may support an augmented reality (AR) experience, where a user of application 300 may be able to virtually see the replacement component in the context of its associated dental equipment. Other related functionality may be provided, such as a video presentation of part replacement, possibly in conjunction with screen 322 as part of the linked documentation. In still other embodiments, links to commence a two-way video session with a support center may be provided, to allow a user of the application 300 to communicate with a service engineer or other expert to assist in repair of the dental equipment, including installing any identified replacement part.

Further, in some embodiments, application 300 may offer related functionality focused around search history and/or favorites. For example, in some embodiments a technician using application 300 may be able to generate multiple request lists for replacement parts, such as a separate list for each of a plurality of customers, e.g. a first list available for a first service appointment, a second list for a subsequent appointment, a third list for a following appointment, etc. In some embodiments employing a CRM, service or sales system, as discussed above with respect to FIG. 2, separate request lists may be associated with each customer. The CRM system may communicate with application 300 to make available, either automatically or manually, the request list of a customer at a scheduled service time. Alternatively or additionally, application 300 may make requests lists from previous, possibly completed, service calls accessible for future reference. Still further, application 300 may allow a technician or other user to create a standing list of commonly used or “favorite” parts, such as a list of expendable or replacement parts most commonly encountered in service calls and/or most commonly required for a given piece of dental equipment.

Finally, although not depicted, in implementations that engage with a CRM, service or sales system, application 300 may provide a front end to at least some of the functionality of the CRM, service or sales system. For example, application 300 may allow a technician or other user to access and/or edit customer information, such as location, contact information, obtain maps and/or guidance to a particular customer's facilities, view summary(ies) of any dental equipment registered to a given customer, locations of a given piece of equipment etc.

FIG. 4 illustrates an example computer device 1500 that may be employed by the apparatuses and/or methods described herein, in accordance with various embodiments. As shown, computer device 1500 may include a number of components, such as one or more processor(s) 1504 (one shown) and at least one communication chip 1506. In various embodiments, one or more processor(s) 1504 each may include one or more processor cores. In various embodiments, the one or more processor(s) 1504 may include hardware accelerators to complement the one or more processor cores. In various embodiments, the at least one communication chip 1506 may be physically and electrically coupled to the one or more processor(s) 1504. In further implementations, the communication chip 1506 may be part of the one or more processor(s) 1504. In various embodiments, computer device 1500 may include printed circuit board (PCB) 1502. For these embodiments, the one or more processor(s) 1504 and communication chip 1506 may be disposed thereon. In alternate embodiments, the various components may be coupled without the employment of PCB 1502.

Depending on its applications, computer device 1500 may include other components that may be physically and electrically coupled to the PCB 1502. These other components may include, but are not limited to, memory controller 1526, volatile memory (e.g., dynamic random access memory (DRAM) 1520), non-volatile memory such as read only memory (ROM) 1524, flash memory 1522, storage device 1554 (e.g., a hard-disk drive (HDD)), an I/O controller 1541, a digital signal processor (not shown), a crypto processor (not shown), a graphics processor 1530, one or more antennae 1528, a display, a touch screen display 1532, a touch screen controller 1546, a battery 1536, an audio codec (not shown), a video codec (not shown), a global positioning system (GPS) device 1540, a compass 1542, an accelerometer (not shown), a gyroscope (not shown), a depth sensor 1548, a speaker 1550, a camera 1552, and a mass storage device (such as hard disk drive, a solid state drive, compact disk (CD), digital versatile disk (DVD)) (not shown), and so forth.

In some embodiments, the one or more processor(s) 1504, flash memory 1522, and/or storage device 1554 may include associated firmware (not shown) storing programming instructions configured to enable computer device 1500, in response to execution of the programming instructions by one or more processor(s) 1504, to practice all or selected aspects of system 100, method 200, and/or process flow 300 described herein. In various embodiments, these aspects may additionally or alternatively be implemented using hardware separate from the one or more processor(s) 1504, flash memory 1522, or storage device 1554.

The communication chips 1506 may enable wired and/or wireless communications for the transfer of data to and from the computer device 1500. The term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. The communication chip 1506 may implement any of a number of wireless standards or protocols, including but not limited to IEEE 802.20, Long Term Evolution (LTE), LTE Advanced (LTE-A), General Packet Radio Service (GPRS), Evolution Data Optimized (Ev-DO), Evolved High Speed Packet Access (HSPA+), Evolved High Speed Downlink Packet Access (HSDPA+), Evolved High Speed Uplink Packet Access (HSUPA+), Global System for Mobile Communications (GSM), Enhanced Data rates for GSM Evolution (EDGE), Code Division Multiple Access (CDMA), Time Division Multiple Access (TDMA), Digital Enhanced Cordless Telecommunications (DECT), Worldwide Interoperability for Microwave Access (WiMAX), Bluetooth, derivatives thereof, as well as any other wireless protocols that are designated as 3G, 4G, 5G, and beyond. The computer device 1500 may include a plurality of communication chips 1506. For instance, a first communication chip 1506 may be dedicated to shorter range wireless communications such as Wi-Fi and Bluetooth, and a second communication chip 1506 may be dedicated to longer range wireless communications such as GPS, EDGE, GPRS, CDMA, WiMAX, LTE, Ev-DO, and others.

In various implementations, the computer device 1500 may be a laptop, a netbook, a notebook, an ultrabook, a smartphone, a computer tablet, a personal digital assistant (PDA), a desktop computer, smart glasses, or a server. In further implementations, the computer device 1500 may be any other electronic device that processes data.

As will be appreciated by one skilled in the art, the present disclosure may be embodied as methods or computer program products. Accordingly, the present disclosure, in addition to being embodied in hardware as earlier described, may take the form of an entirely software embodiment (including firmware, resident software, micro-code, etc.) or an embodiment combining software and hardware aspects that may all generally be referred to as a “circuit,” “module” or “system.” Furthermore, the present disclosure may take the form of a computer program product embodied in any tangible or non-transitory medium of expression having computer-usable program code embodied in the medium.

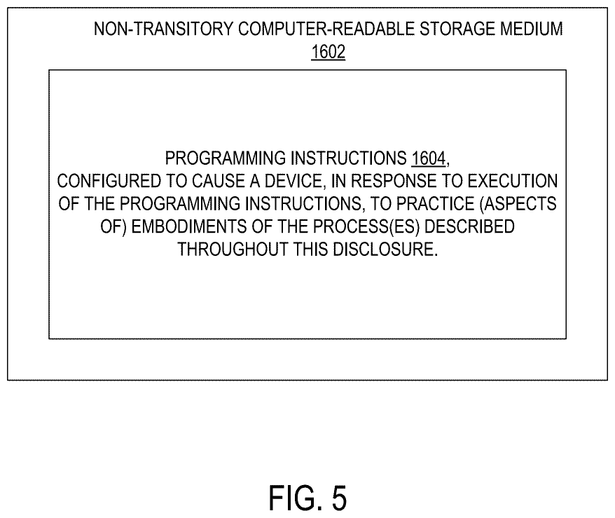

FIG. 5 illustrates an example computer-readable non-transitory storage medium that may be suitable for use to store instructions that cause an apparatus, in response to execution of the instructions by the apparatus, to practice selected aspects of the present disclosure. As shown, non-transitory computer-readable storage medium 1602 may include a number of programming instructions 1604. Programming instructions 1604 may be configured to enable a device, e.g., computer 1500, in response to execution of the programming instructions, to implement (aspects of) method 200 and/or process flow 300 described above. In alternate embodiments, programming instructions 1604 may be disposed on multiple computer-readable non-transitory storage media 1602 instead. In still other embodiments, programming instructions 1604 may be disposed on computer-readable transitory storage media 1602, such as, signals.

Any combination of one or more computer usable or computer readable medium(s) may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, RF, etc.

Computer program code for carrying out operations of the present disclosure may be written in any combination of one or more programming languages, including an object oriented programming language such as Java, Smalltalk, C++, Objective-C, or the like, platforms and/or scripting environments such as React-Native, ClojureScript, NativeScript, or the like, and/or conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowchart and/or block diagram block or blocks.

It will be apparent to those skilled in the art that various modifications and variations can be made in the disclosed embodiments of the disclosed device and associated methods without departing from the spirit or scope of the disclosure. Thus, it is intended that the present disclosure covers the modifications and variations of the embodiments disclosed above provided that the modifications and variations come within the scope of any claims and their equivalents.

What is claimed is:

1. A method, comprising:
receiving an image of a component of a dental equipment, the image depicting at least a portion of the component;
searching, using the image, into a database of possible matching components;
identifying, from the database, one or more possible matching components corresponding to the component depicted in the image; and
either:
generating, from the one or more identified possible matching components, a list of replacement parts, or
providing, from the one or more parts of the component, documentation related to the one or more possible matching components and the dental equipment.

2. The method of claim 1, wherein identifying, from the database, one or more possible matching components comprises providing the image to a machine learning algorithm for identification.

3. The method of claim 1, further comprising making the list of replacement parts available to an application programming interface (API), the API accessible by an equipment manufacturer, sales and service dealer, or parts provider.

4. The method of claim 1, further comprising:
accessing, over a network, the dental equipment; and
receiving, over the network, diagnostic information of the dental equipment.

5. The method of claim 4, wherein accessing the dental equipment comprises registering the dental equipment using a wireless signal or an optical marker.

6. A non-transitory computer-readable medium comprising instructions that, when executed by an apparatus, cause the apparatus to:
capture an image of at least a portion of a component of a dental equipment;
provide the image to a machine-learning service; and
receive, from the machine learning service, a list of one or more likely components corresponding to component depicted in the image, and
either:
generate, from the list of one or more identified likely components, a list of replacement parts, or
provide, for at least one of the one or more likely components, documentation related to the at least one component and the dental equipment.

7. The computer-readable medium of claim 6, wherein the instructions are to further cause the apparatus to:
interface with the dental equipment; and
view diagnostic information of the dental equipment.

8. The computer-readable medium of claim 7, wherein the instructions are to further cause the apparatus to interface with the dental equipment by first registering the dental equipment using a wireless signal or an optical marker.

9. The computer-readable medium of claim 6, wherein the instructions are to further cause the apparatus to provide documentation related to the at least one likely component by providing one or more links to download a copy of the documentation.

10. The computer-readable medium of claim 6, wherein the instructions are to further cause the apparatus to make the list of possible replacement parts available to an application programming interface (API), the API accessible by an equipment manufacturer, sales and service dealer, or parts provider.

11. The computer-readable medium of claim 6, wherein the apparatus is a mobile device.

12. The computer-readable medium of claim 11, wherein the instructions comprise an application to be executed on the mobile device.

13. A system, comprising:
a dental equipment; and
a computing device;
wherein the computing device is to:
communicate with the dental equipment to obtain diagnostic information from the dental equipment,
capture an image of at least part of a component of the dental equipment, search, with the image of the at least part of the component, a database of components, and
provide access to one or more documents relevant to the dental equipment.

14. The system of claim 13, wherein the database of replacement parts is to search using a machine learning process.

15. The system of claim 14, wherein the machine learning process is hosted on a remote server in communication with the computing device, and to search with the image of the at least part of the component comprises transmitting the image to the remote server.

16. The system of claim 13, wherein the computing device is to communicate with the dental equipment by first registering the dental equipment using a wireless signal or an optical marker.

17. The system of claim 13, wherein the computing device is to determine, based on the search, a list of possible replacement parts for the component.

18. The system of claim 17, wherein the computing device is further to provide, with the list of possible replacement parts, access to one or more documents relevant to at least one of the possible replacement parts.

19. The system of claim 17, wherein the computing device is further to make the list of possible replacement parts available to an application programming interface (API), the API accessible by a parts provider.

20. The system of claim 13, wherein the computing device is a mobile device.
